(19) United States
(12) Patent Application Publication
(10) Pub. No.: US 2022/0252613 A1
(43) Pub. Date: Aug. 11, 2022

(54) IONISATION CONTROL

(71) Applicant: The Binding Site Group Limited, Birmingham (GB)

(72) Inventors: Stephen HARDING, Birmingham (GB); Gregg WALLIS, Birmingham (GB); Jamie ASHBY, Birmingham (GB); Nia MARROTT, Birmingham (GB); Simon NORTH, Birmingham (GB)

(21) Appl. No.: 17/629,747

(22) PCT Filed: Jul. 23, 2020
(86) PCT No.: PCT/GB2020/051758

§ 371 Date: Jan. 24, 2022

(30) Foreign Application Priority Data
Jul. 26, 2019 (GB) 1910697.0

(51) Int. Cl. G01N 33/68; G16B 15/00; G01N 33/96

(52) U.S. Cl. CPC G01N 33/6851; G01N 33/96; G16B 15/00

(57) ABSTRACT

An elution buffer for eluting one or more predetermined analytes from one or more analyte-specific antibodies or fragments thereof or for eluting one or more predetermined antibodies or fragments from a target antigen, wherein: the elution buffer has a pH of 1 to 5; and the elution buffer comprises a predetermined amount of an acid stable mass spectrometry ionisation control protein. The use of the elution buffer in the detection and quantifying of analytes, for example by mass spectrometry is also described.

The invention relates to an elution buffer comprising a predetermined amount of an acid stable ionisation control for use in mass spectrometry, kits containing such buffers and methods of producing such buffers and kits.

### BACKGROUND

Protein profiling by mass spectrometry has significant clinical utility in in vitro diagnostics; however, analytical reproducibility remains a potential issue and peak intensity and m/z values can vary significantly between experiments.

Current approaches to controlling for analytical variability and reproducibility to enable routine use in in vitro diagnoses of human diseases comprise automated sample processing, extensive prefractionation strategies, immunocapture, prestructured target surfaces, standardized matrix (co)crystallization, improved MALDI-TOF mass spectrometry (MS) instrument components, internal standard peptides, quality-control samples, replicate measurements, and algorithms for normalization and peak detection (Albrethsen, J., 2007; Clin Chem, 53(5) 852-858).

However, these methods are subject to other factors in addition to crystal formation between the matrix and the sample and thus cannot accurately reflect spotting, crystallisation and ionisation.

Previous attempts to control for inter and intra-experimental variability in MALDI-TOF MS have typically utilised internal calibrating peptides with comparable physicochemical properties to a protein of interest, spiked into a sample at varying concentrations before comparison of the ion intensities of the calibrating peptide and the analyte. However, problems with this approach have included variability in peak intensity. Combining controls with iterative algorithms and/or performing analyses of replicates have been suggested as possible solutions to compensate for some of the analytical variation over time and improving reproducibility of protein profiling by MALDI-TOF MS (Albrethsen, J., Clin Chem 2007; 53(5): 852-858).

For example, one study utilised a method whereby a synthetic peptide with the same primary sequence as a specific analyte was spiked into a blood sample (Yi, J et al., Methods Mol Biol 2011; 728: 161-75).

The use of an internal control for matrix-assisted laser desorption/ionization time-of-flight mass spectrometry analysis was also shown to improve the sensitivity of determining the concentration of bacteria in a sample. Addition of cytochrome C as an internal control reduced signal intensity by 20-30% in samples with high concentrations of bacteria but improved signal intensity at some low concentrations of bacteria. In this case, the protein was spiked into the matrix, which was then premixed in a 2:1 matrix to analyte ratio (Gantt, S L et al., J Am Soc Mass Spectrom 1999; 10(11):1131-7).

In another example, ion suppression effects in atmospheric pressure matrix-assisted laser desorption/ionization were investigated by spiking angiotensin II analogue as an internal standard into all fractions of a 384 Prespotted AnchorChip. (Li, G et al., Rapid Commun Mass Spectrom 2019; 33(4): 327-335). Signal intensities were then normalised according to the control, followed by peak clustering analysis. Lower intensity peaks had reproducibility equivalent to higher intensity peaks.

An example of other attempts to adjust for variability in quantitation between samples include post-analysis adjustment by outlier removal and baseline removal by intensity scaling (Neubert et al., J Proteome Res 2008; 7(6) 2270-9).

An example of in vitro diagnostic uses for which mass spectrometry methods have significant utility is in relation to a number of proliferative diseases associated with antibody producing cells.

Antibody molecules (also known as immunoglobulins) have a twofold symmetry and typically are composed of two identical heavy chains and two identical light chains, each containing variable and constant domains. The variable domains of the heavy and light chains combine to form an antigen-binding site, so that both chains contribute to the antigen-binding specificity of the antibody molecule. The basic tetrameric structure of antibodies comprises two heavy chains covalently linked by a disulphide bond. Each heavy chain is in turn attached to a light chain, again via a disulphide bond. This produces a substantially “Y”-shaped molecule.

In many such proliferative diseases a plasma cell proliferates to form a monoclonal tumour of identical plasma cells. This results in production of large amounts of identical immunoglobulins and is known as a monoclonal gammopathy.

Diseases such as myeloma and primary systemic amyloidosis (AL amyloidosis) account for approximately 1.5% and 0.3% respectively of cancer deaths in the United Kingdom. Multiple myeloma is the second-most common form of haematological malignancy after non-Hodgkin lymphoma. In Caucasian populations the incidence is approximately 40 per million per year. Conventionally, the diagnosis of multiple myeloma is based on the presence of excess monoclonal plasma cells in the bone marrow, monoclonal immunoglobulins in the serum or urine and related organ or tissue impairment such as hypercalcaemia, renal insufficiency, anaemia or bone lesions. Normal plasma cell content of the bone marrow is about 1%, while in multiple myeloma the content is typically greater than 10%, frequently greater than 30%, but may be over 90%.

AL amyloidosis is a protein conformation disorder characterised by the accumulation of monoclonal free light chain fragments as amyloid deposits. Typically, these patients present with heart or renal failure but peripheral nerves and other organs may also be involved.

There are a number of other diseases which can be identified by the presence of monoclonal immunoglobulins within the blood stream, or indeed urine, of a patient. These include plasmacytoma and extramedullary plasmacytoma, a plasma cell tumour that arises outside the bone marrow and can occur in any organ. When present, the monoclonal protein is typically IgA. Multiple solitary plasmacytomas may occur with or without evidence of multiple myeloma. Waldenström's macroglobulinaemia is a low-grade lymphoproliferative disorder that is associated with the production of monoclonal IgM. There are approximately 1,500 new cases per year in the USA and 300 in the UK. Serum IgM quantification is important for both diagnosis and monitoring. B-cell non-Hodgkin lymphomas cause approximately 2.6% of all cancer deaths in the UK and monoclonal immunoglobulins have been identified in the serum of about 10-15% of patients using standard electrophoresis methods. In B-cell chronic lymphocytic leukaemia monoclonal proteins have been identified by free light chain immunoassay.

Additionally, there are so-called MGUS conditions. These are monoclonal gammopathy of undetermined significance. This term denotes the unexpected presence of a monoclonal intact immunoglobulin in individuals who have no evidence of multiple myeloma, AL amyloidosis, Waldenström's macroglobulinaemia, etc. MGUS may be found in 1% of the population over 50 years, 3% over 70 years and up to 10% over 80 years of age. Most of these are IgG- or IgM-related, although more rarely IgA-related or bi-clonal. Although most people with MGUS die from unrelated diseases, MGUS may transform into malignant monoclonal gammopathies.

In at least some cases for the diseases highlighted above, the diseases present abnormal concentrations of monoclonal immunoglobulins or free light chains. Where a disease produces the abnormal replication of a plasma cell, this often results in the production of more immunoglobulins by that type of cell as that “monoclone” multiplies and appears in the blood.

A sensitive assay has been developed that can detect the free kappa light chains and separately, the free lambda light chains. This method uses a polyclonal antibody directed towards either the free kappa or the free lambda light chains. The possibility of raising such antibodies was also discussed as one of a number of different possible specificities, in WO 97/17372. This document discloses methods of tolerising an animal to allow it to produce desired antibodies that are more specific than prior art techniques could produce. The free light chain assay uses the antibodies to bind to free lambda or free kappa light chains. The concentration of the free light chains is determined by nephelometry or turbidimetry.

The characterisation of the amount or types of free-light chains (FLC), heavy chain or subclasses, or light chain-type bound to heavy chain class or subclass, is important in a wide range of diseases including B cell diseases such as multiple myeloma and other immune mediated diseases including B-cell diseases such as monoclonal gammopathies (where Multiple myeloma is an example), and other immune mediated diseases, including both hypergammaglobulinaemias and hypogammaglobulinaemieas.

WO2015/154052, incorporated herein in its entirety, discloses methods of detecting immunoglobulin light chains, immunoglobulin heavy chains, or mixtures thereof, using MS. Samples comprising immunoglobulin light chains, heavy chains or mixtures thereof are immunopurified and subjected to mass spectrometry to obtain a mass spectrum of the sample. This can be used to detect monoclonal proteins in samples from patients. It can also be used to fingerprint, isotype and identify monoclonal antibodies.

MS is used to separate, for example, lambda and kappa chains in the sample by mass and charge. It may also be used to detect the heavy chain and light chain component of immunoglobulins, by, for example reducing the disulphide bonds between heavy and light chains using a reducing agent. MS is also described in WO 2015/131169, herein incorporated in its entirety.

The purification of immunoglobulins in a sample in diagnostic procedures, typically uses antibodies against whole antibodies and/or free light chains, such as anti-IgG, anti-IgA, anti-IgM, anti-IgD, anti-IgE, anti-total kappa, anti-total lambda antibodies or anti-free light chain antibodies, such as anti-free κ or anti-free λ light chain antibodies. It is important to have a calibrator to ensure that the purification and detection process is carried out correctly.

WO2017/144900 describes a number of controls that utilise either heavier versions of the analyte to be detected or a monoclone of the analyte to be detected. That is, for example, IgA may be quantified in comparison to a predetermined amount of a heavier IgA kappa.

This is because it was expected that different proteins would crystallise on the mass spectrometry matrix at different rates. This would mean that when the matrix is sampled by mass spectrometry, different amounts of the control protein and the analyte would be detected. Moreover, their ionisation rates were expected to be different. This would lead to inconsistencies in the amount of immunoglobulins apparently detected. Additionally, the problem of analytical reproducibility of mass spectrometry-based methods means that peak intensity can vary significantly between experiments and mass drift can occur which could affect the recorded m/z value. MALDI-TOF ionisation, for example, is dependent on a spot to spot variable process of crystal formation between the matrix (e.g. HCCA) and the sample.

The authors have surprisingly found that analytical variability in ionisation for mass spectrometry can be controlled by using an independent marker within an acidic elution buffer following immunoprecipitation and prior to spotting.

### SUMMARY

Provided herein is an elution buffer for eluting one or more predetermined analytes from one or more analyte-specific antibodies or fragments thereof or for eluting one or more predetermined antibodies or fragments from a target antigen, wherein:
the elution buffer has a pH of 1 to 5, more preferably pH 1 to 3, or even more preferably pH 1.5 to 3.0; and the elution buffer comprises a predetermined amount of an acid stable mass spectrometry ionisation control protein.

The elution buffer may be used to elute, for example analyte bound to antibodies attached to a substrate. Alternatively a target antigen may be attached to a substrate and antigen specific antibodies or fragments eluted from the target antigen

Such elution buffers are used to release analyte bound to the analyte specific antibodies. Having the ionisation control included in the buffer allows them to be provided by a supplier and reduces user errors caused by the user having to separately measure or prepare the amount of ionisation control material to use.

The ionisation control can also be used as a “lock mass spectra calibrator” in mass spectrometry methods including, for example, both MALDI and electrospray mass spectrometry. Such lock mass spectra calibrators are ions having a known m/z value derived from the ionisation control which permits real time recalibration within a spectrum by correction of m/z shifts arising from instrumental and intra MALDI-target plate drifting.

The sample containing the analyte to be analysed may be a biological sample such as blood, saliva, serum, plasma, cerebrospinal fluid or urine, more typically blood, serum or plasma.

The sample may be from a subject exhibiting hypogammaglobulinaemia or hypergammaglobulinaemia. The subject may have a proliferative disease associated with antibody producing cells, such as a monoclonal gammopathy. These include myeloma and primary systemic amyloidosis, plasmacytomas, Waldenström's macroglobulinemia, and MGUS.

The ionisation control protein is selected to be compatible with the predetermined analyte.

The ionisation control protein may be substantially stable in the elution buffer for at least 30 days, more preferably at least 60 days, typically at least 4 months or at least 6 months.

Long term storage of an unsuitable control may lead to physical stability issues that could lead to the protein precipitating in the elution buffer or otherwise affected and so resulting in poor crystallisation on the target or poor ionisation, both of which could affect the m/z peak height or area.

Damage to the control itself that results in a change in mass or ionisation states could also alter the measured m/z.

The control protein may be stable at, for example, 22° C. or lower, or 4° C. It may be pH, UV or light stable.

At least one mass spectrometry m/z peak value of the ionisation control protein may be substantially stable as defined above.

The ionisation control protein may be selected to have at least one mass spectrometry peak having an m/z value which does not substantially overlap with a mass spectrometry peak of the or each predetermined analyte. It is typically selected to ionise consistently and typically not substantially affect the intensity of the mass spectrometry signal.

The ionisation control protein may be selected to have at least one mass spectrometry m/z peak value within a predetermined mass spectrometry window, or the m/z range looked at by the mass spectrometer, used for the detection or quantification of one or more peaks from the at least one predetermined analyte.

A sample may be treated with a reducing agent, typically after elution but prior to performing mass spectrometry. This is particularly useful where the immunoglobulin light chains in the sample, are bound to heavy chains. The use of a reducing agent decouples the light chains from the heavy chains and allows the light chains to be detected separately by the mass spectrometry. Reducing agents may also be used to separate other analyte proteins to separate subunits where present.

Decoupling can be achieved by treating the total immunoglobulins with a reducing agent, such as DTT (2, 3 dihydroxybutane-1,4-dithiol), DTE (2, 3 dihydroxybutame-1, 4-dithiol), thioglycolate, cysteine, sulphites, bisulfites, sulphides, bisulfides, TCEP (tris (2-carboxyethyl) phosphine), 2-mercaptoethanol, and salt forms thereof. In some embodiments, the reducing step is performed at elevated temperature, e.g. in a range from about 30° C. to about 65° C., such as about 55° C., in order to denature the proteins.

The decoupling step is usually carried out after immunopurification or other enrichment of the immunoglobulins in the sample or as part of an elution step after immunopurification of the sample.

The antibodies used in immunopurification may be intact antibodies or fragments thereof, such as Fab, F(ab) and F(ab′)² fragments, or single chain antibodies. The antibodies or fragments thereof may be cross-linked, for example as described in WO2017144903 herein incorporated in its entirety.

Any acidic buffer (pH 1 to 5, more preferably pH 1 to 3, or pH 1.5 to 3), could be used as long as it does not interfere with the mass spectrometry, such as MALDI-TOF, ionisation process.

The elution buffer may comprise organic acids such as citric acid, acetic acid, formic acid, uric acid, propionic acid and inorganic acids such as hydrochloric acid. Acidic buffers or solutions that contains salts, may be avoided, especially at higher concentrations as at high concentrations these may interfere with ionisation or crystallisation.

For example, the elution buffer of the invention may comprise an elution buffer selected from
(a) 5% v/v acetic acid in water;
(b) 0.1 M glycine pH 2.0-3.0, or 0.2 M glycine pH 2-6

A buffer comprising 5% acetic acid preferably has a pH of approximately 2.

The elution buffer may comprise 1 to 100 ng/μl of ionisation control protein, more preferably 1 to 10 ng/μl.

A reducing agent may be used in combination with the elution buffer, and may further comprise tris(2-carboxyethyl)phosphine, dithiothreitol, 2-mercaptoethanol, or cysteine. The reducing agent may be preweighed, or provided to supply a final concentration in the range of 10-100 mM, or more preferably approximately 20 mM.

The ionisation control protein may comprise at least 30 amino acids or at least 50 amino acids and/or may have a mass of at least 3 kDa or for eluting one or more predetermined antibodies or fragments from a target antigen kDa.

The ionisation control protein advantageously has different mass range, or ion gates, or have multiple charge states to enable use within the assay window of the analyte. The ionisation control protein or peptide may be naturally-occurring or synthetic.

Suitable proteins for use as an ionisation control may comprise aprotinin, α1 acid glycoprotein, μ2 glycoprotein, or prealbumin (also referred to as transthyretin). More preferably, the ionisation control may comprise aprotinin or transthyretin. Aprotinin is a serine protease inhibitor derived from the bovine pancreas. It is readily available from commercial sources as both a pure protein and a drug; TRASYLOL. (CAS Number: 9087-70-1, molar mass 6511.5 Da. UniProtKB accession number P00974. Isoelectric point pH 10.5). Stable in neutral or acidic media at high temp. Transthyretin (TTR, prealbumin or TBPA) is a transport protein found in serum and cerebrospinal fluid that carries the thyroid hormone thyroxin and retinol binding protein bound to retinol. It is a 55 kDa homotetramer or a dimer of dimers quaternary structure. The human protein has the UniProtKB accession number P02766.

However, other substantially acid stable proteins could be used in different mass ranges (ion gates) or where one or more of the protein charge states are suitable for use in a particular m/z assay window. For example, in an assay window of m/z 5-30 kDa, α1 acid glycoprotein (+1˜21560), β2 glycoprotein I (+1 36255) or pre albumin monomer (+1˜13760) would be suitable.

Targeting larger mass windows e.g. dual ion gating approach, or wide mass windows is possible through utilisation of an additional plate in the instrument and inversion of gates.

Also provided herein is a kit for use in the analysis by mass spectrometry of one or more analytes comprising an elution buffer as defined above and one or more analyte specific antibodies or fragments thereof specific for the one or more predetermined analytes.

The analyte or antigen specific antibody may be a protein or peptide, more preferably a serum protein or peptide.

Antigen specific antibodies include anti-streptolysin O, anti-tetanus toxoid immunoglobulin, Haemophilus influenzae-specific immunoglobulin, Diptheria toxoid specific immunoglobulin, Streptococcus pneumoniae specific immunoglobulin, Salmonella typhi-specific immunoglobulin or Varicella zoster virus-specific immunoglobulin.

If the analyte is a serum protein, the serum protein may comprise one or more of a complement protein, for example the serum protein may comprise one or more of complement protein components, such as C1, C2, C3, C4, or components thereof, for example components C3a, C3b, C3c.

The serum protein may comprise an immunoglobulin or fragment thereof, albumin, β2-microglobin, α1-microglobin, cystatin C, a microalbumin, α1-acid glycoprotein, α1-antitrypsin, α2-macroglobin, anti-streptolysin O, anti-tetanus toxoid immunoglobulin, apolipoprotein A, apolipoprotein B, caeruloplasmin, C-reactive protein, haptoglobin, prealbumin, rheumatoid factor or total serum protein transferrin.

The analyte may be a monoclonal antibody, such as a therapeutic monoclonal antibody Analyte specific antibodies that may be comprised within the kit may be one or more of anti-IgA, anti-IgG, anti-IgM, anti-IgD, anti-IgE, anti-total light chain, anti-free light chain, anti-lambda light chain, anti-kappa light chain, anti-lambda free light chain, anti-kappa free light chain, anti-heavy chain subclass, anti-heavy chain class-light chain type or anti-heavy chain subclass-light chain type specific antibodies; more preferably anti-IgG, anti-IgA, anti-IgM, anti-kappa and/or anti-lambda-specific antibodies.

The antibodies or fragments thereof specific for the one or more predetermined analytes, may further be bound to a substrate; for example, the antibodies or fragments thereof may be bound to latex beads. Target antigens may also be attached to a substrate such as latex beads.

The kit may further comprise a predetermined amount of a control analyte.

The kit may comprise one or more of a sample diluent buffer, an immunocapture reagent or bead, a wash buffer, an elution buffer containing an optional reducing agent, a mass spectrometry matrix, a mass spectrometry matrix solvent, a MALDI target and a mass spectrometer mass calibrator.

The reducing agent of the kit may comprise tris(2-carboxyethyl)phosphine, dithiothreitol, 2-mercaptoethanol, or cysteine and may be defined as above.

The reducing agent is preferably pre-weighed, or otherwise provided to supply a final concentration in the range of 10-100 mM, or more preferably approximately 20 mM.

The kit may additionally comprise a standard serum protein control. For example, the kit may comprise antibodies that are anti-human specific antibodies.

Also provided herein is a method of detecting or quantifying an analyte comprising immunopurifying a predetermined analyte, eluting the analyte with an elution buffer according to the invention and detecting the analyte and the ionisation control protein by mass spectrometry.

The method is not limited to any particular method of mass spectrometry; however, the mass spectrometry method may comprise liquid chromatography mass spectrometry (LC-MS) or MALDI-TOF mass spectrometry. More preferably, the method of mass spectrometry may comprise MALDI-TOF mass spectrometry.

An immunoassay to which the invention applies has three main steps; 1) immunocapture of an analyte, 2) elution of the analyte, 3) optional reduction of the analyte, and 4) spotting of the analyte onto a MALDI-TOF target plate.

The invention provides that an ionisation control protein may be included within the reagent used for step 2) so that the control and analyte are combined prior to step 3), and are advantageously spotted together in step 4. This is important since the ionisation control is used to control for variability in step 3 and subsequent ionisation within the MALDI-TOF mass spectrometer.

The method may further provide the use of a kit in accordance with the invention, for use in the analysis by mass spectrometry of one or more analytes comprising an elution buffer according to any preceding claim and one or more analyte specific antibodies or fragments thereof specific for the one or more predetermined analytes.

Also provided herein is a method of producing an elution buffer in accordance with the invention, wherein the elution buffer is for eluting one or more predetermined analytes from one or more analyte-specific antibodies or fragments thereof, wherein:
the elution buffer has a pH of 1 to 6, more preferably pH 2 to 6, more preferably pH 1 to 4, or even more preferably pH 1.5 to 3.0; and the elution buffer comprises a predetermined amount of an acid stable mass spectrometry ionisation control protein.

The method of producing an elution buffer in accordance with the invention comprises:
(a) identifying the analyte;
(b) identifying the m/z of at least one peak for the ionisation control compared to the m/z of one or more expected peaks of the analyte;
(c) identifying ionisation control proteins having the m/z range and acid stability.

A computer implemented method comprising imputing an analyte, comparing one or more m/z peaks for the analyte with the m/z peak of a plurality of potential ionisation control proteins having acid stability, and outputting the identification of one or more ionisation control proteins having the m/z range and acid stability for the analyte.

### DESCRIPTION OF FIGURES

The invention will now be described by way of example only with reference to the following figures.

FIG. 1 is an example of a MALDI-TOF mass spectrum showing the mass distribution of analyte (kappa light chain (k)) following elution with acetic acid containing aprotinin. A single aprotinin peak is observed (+1 charge) which does not interfere with the analyte (kappa light chain) peaks. Ion charge states are given in parentheses.

FIG. 2 shows that the relative ionisation control protein signal remains stable in the presence and absence of analyte. The MALDI-TOF mass spectrum of aprotinin obtained in the absence of analyte (black line) does not significantly differ from the aprotinin spectrum containing kappa light chains (grey line and inset) derived from normal human serum (NHS).

FIG. 3 shows that aprotinin remains stable in 5% acetic acid. Kappa light chains were periodically eluted with 5% acetic acid containing aprotinin that had been stored at 22° C. MALDI-TOF mass spectra were acquired at each time point and the aprotinin and kappa light chain (+2) peak areas were determined (±standard deviation). No depreciation in signal was observed for either protein over an 8 week period.

FIG. 4 shows that the analyte signal relative to aprotinin as an ionisation control stays stable over time. 5% Acetic acid containing aprotinin was stored at 22° C. and used periodically to elute kappa light chains. MALDI-TOF mass spectra were acquired at each time point and the aprotinin and kappa light chain (+2) peak area ratio were determined (±standard deviation). No appreciable change in peak area ratio was observed over an 8 week period.

FIG. 5 Transthyretin (TTR) as an MALDI-TOF ionisation control. MALDI-TOF mass spectrum showing the mass distribution of TTR following elution with acetic acid, with and without mixing with polyclonal IgG (FIGS. 5A and B). A TTR peak is observed at 13827 m/z and at 6914 m/z neither of which interferes with any of the lambda or kappa polyclonal light chain peaks. The signal intensity of the TTR ionisation control peak is unchanged in the presence or absence of the analyte (B). The TTR signal peaks also do not overlap with those of aprotinin (C), nor a glycosylated kappa free light chain (of greater mass) (D). Ion charge states are given in parentheses.

An elution buffer was prepared comprising 2 ng ml⁻¹ of aprotinin as an ionisation control for mass spectrometry in 5% acetic acid containing 20 mM tris(2-carboxyethyl) phosphine (TCEP) reducing agent. 5% acetic acid was used to both elute the analyte from the immunocapture bead and to simultaneously facilitate separation of immunoglobulin heavy chain and light chain. 20 mM TCEP was used acid an acid stable reducing agent to break the disulphide bonds holding intact immunoglobulins together.

A normal human serum sample (NHS) was diluted 1:10 and captured, (as per step 1 above), using a paramagnetic microparticle containing antibodies specific for human kappa immunoglobulin light chains. This was eluted with an acidic buffer solution containing both reducing agent and aprotinin (as an ionisation control). The elution was subsequently spotted, in a sandwich with MALDI matrix (HCCA) onto a MALDI-TOF target plate and dried. Mass spectra were acquired in positive ion mode covering the m/z range of 5000 to 30,000 which includes the singly charged (+1, m/z22705), doubly charged (+2, m/z 11353) and triply charged (+3, m/z 7569) ions of the analyte (human kappa light chains; Table 1).

TABLE 1
Mass spectra acquired in positive ion mode

| Protein | +1 | +2 | +3 |
|---|---|---|---|
| Aprotinin | 6512 | 3257 | 2171 |
| | (assuming | (assuming | (assuming |
| | [M + H]⁺) | [M + 2H]²⁺) | [M + 3H]³⁺) |
| Transthyretin | 13827 | 6914 | Not |
| | (assuming | (assuming | determined |
| | [M + H]⁺) | [M + 2H]²⁺) | |
| Polyclonal | 22705 | 11353 | 7569 |
| kappa LC | (assuming | (assuming | (assuming |
| | [M + H]⁺) | [M + 2H]²⁺) | [M + 3H]³⁺) |
| Polyclonal | 23440 | 11720.5 | 7814 |
| lambda | (assuming | (assuming | (assuming |
| LC | [M + H]⁺) | [M + 2H]²⁺) | [M + 3H]³⁺) |

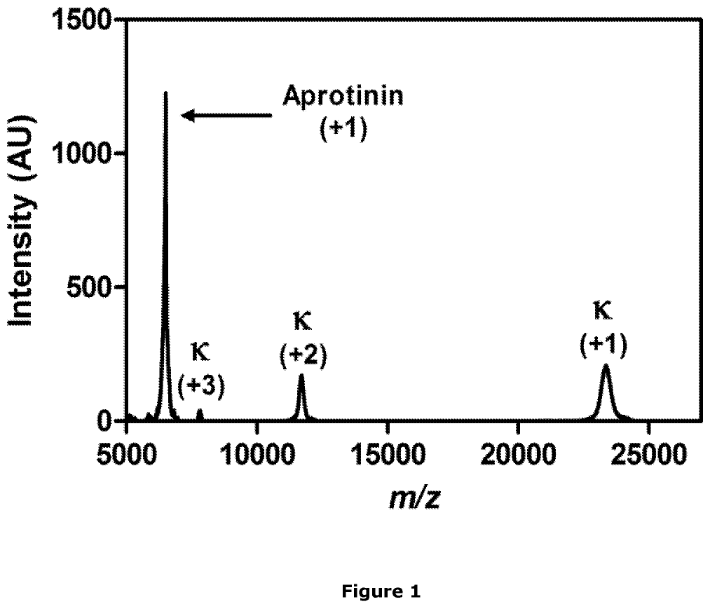

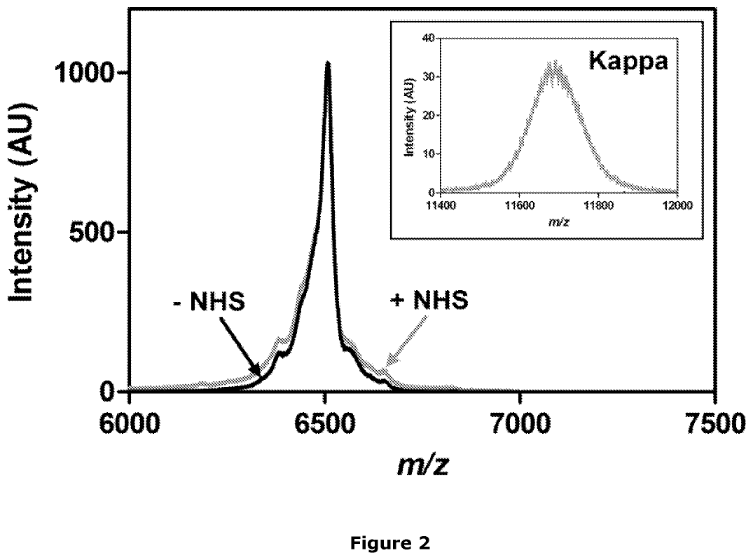

The aprotinin intensity signal is clearly seen in FIG. 1 as a distinct peak of m/z 6512 that does not interfere or overlap with any of the three peaks of the analyte. To show that the aprotinin signal is independent of the presence of the analyte, it was analysed in the presence (+NHS) and absence (−NHS) of the latter. FIG. 2 illustrates that the aprotinin ionisation control signal intensity is the same in either case.

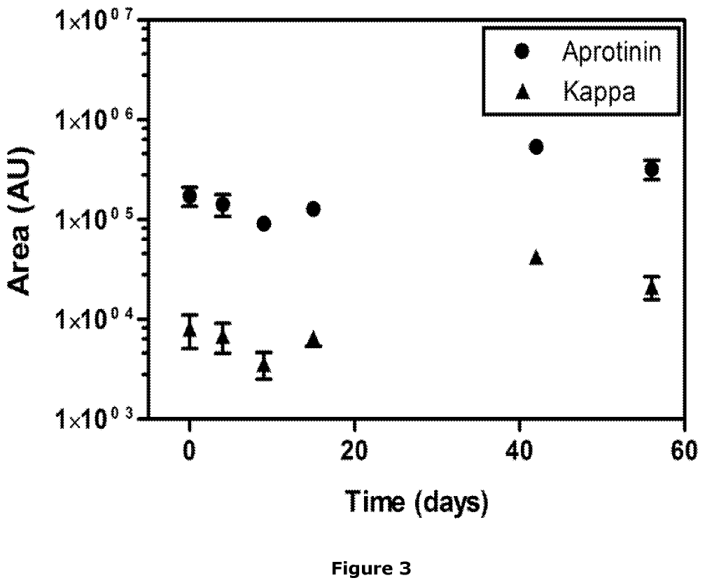

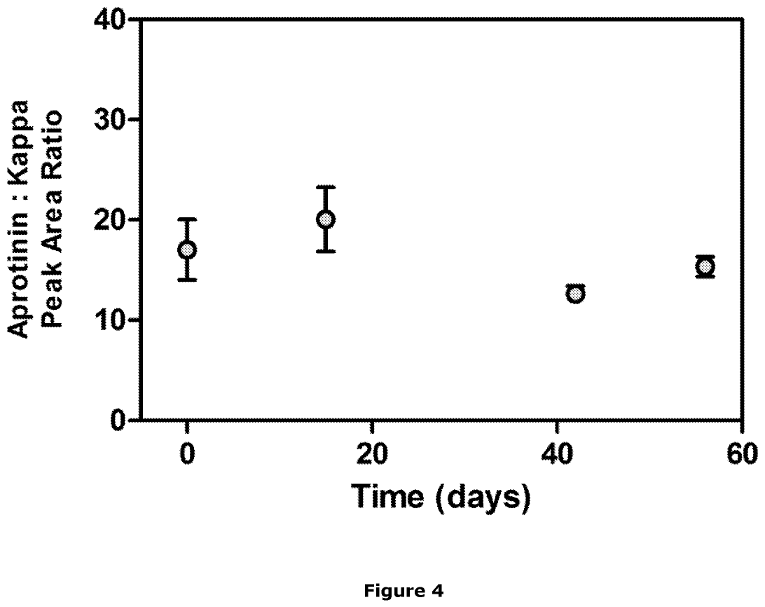

To investigate the stability of the ionisation control in acidic conditions, individual 50 ml aliquots of the formulation (5% acetic acid supplemented with 2 ng ml⁻¹ aprotinin) were stored at 22° C. Individual aliquots were removed at regular intervals, supplemented with the reducing agent (TCEP) and then used to elute the analyte from an anti-kappa microparticle for MALDI-TOF analysis. The mass-spectrometric peak areas obtained for both the kappa analyte and aprotinin are shown in FIG. 3. The peak areas for both vary during the experiment. This variation is due to known MALDI spot-to-spot sample inconsistency, but there is no degradation in either analyte or aprotinin signal over a 60 day period at 22° C. By extrapolation this indicates that aprotinin is stable in acidic conditions for at least 6 months when stored at 4° C. (using the Arrhenius equation). When the stability data is expressed as a ratio of analyte signal peak to ionisation control signal peak, the variability is considerably minimised (FIG. 4). This illustrates the use of the aprotinin (as an ionisation control) to overcome ionisation differences between different MALDI-TOF acquisitions.

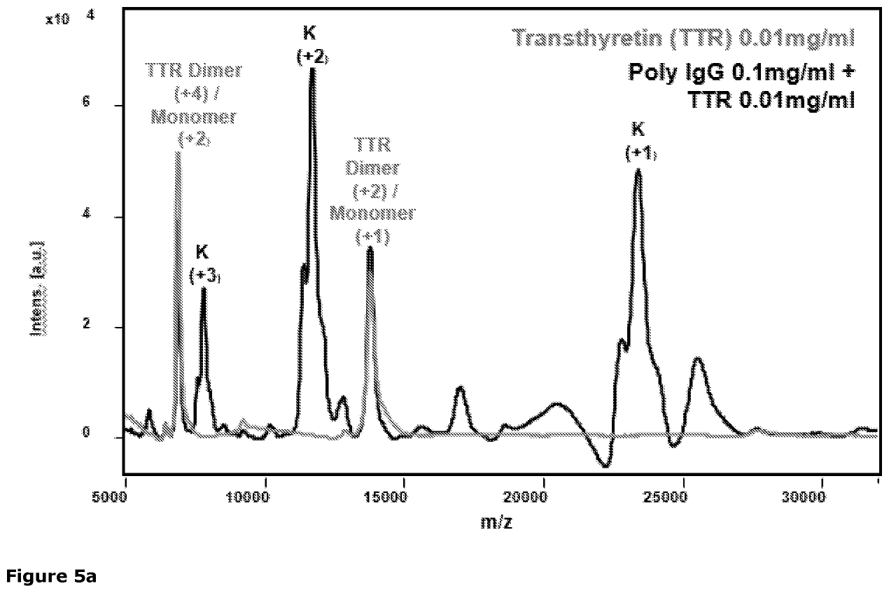

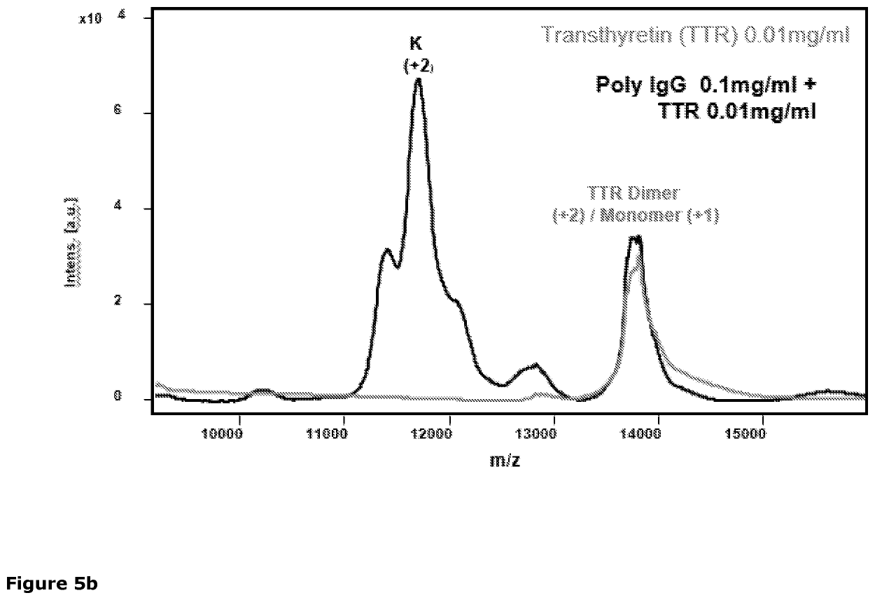

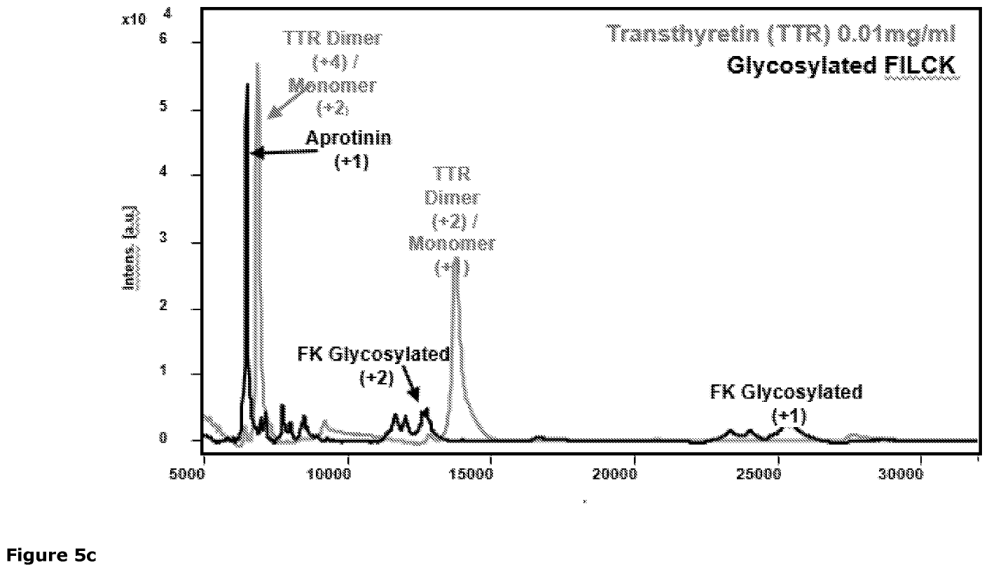

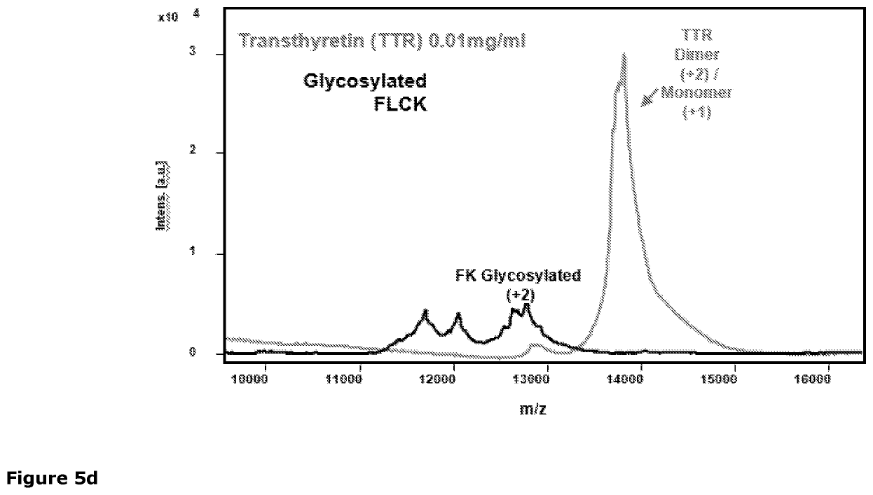

FIG. 5 shows another example of an ionisation control, transthyretin. MALDI-TOF mass spectrum were produced showing the mass (m/z) distribution of TTR (0.01 mg/ml) in elution buffer in the presence or absence of 0.1 mg/ml polyclonal IgG (FIGS. 5A and B). A TTR monomer peak is observed at 13827 m/z (+1 charge state) and at 6914 m/z (+2 charge state) neither of which interferes with any of the lambda or kappa polyclonal light chain peaks from the IgG. The signal intensity of the TTR ionisation control peak is unchanged in the presence or absence of the analyte (FIG. 5B). The TTR signal peaks also do not overlap with those of aprotinin (FIG. 5C), nor a glycosylated kappa free light chain (of greater m/z) (FIG. 5D).

1. An elution buffer for eluting one or more predetermined analytes from one or more analyte-specific antibodies or fragments thereof or for eluting one or more predetermined antibodies or fragments from a target antigen, wherein:
the elution buffer has a pH of 1 to 5; and the elution buffer comprises a predetermined amount of an acid stable mass spectrometry ionisation control protein.

2. An elution buffer according to claim 1, wherein the ionisation control protein is substantially stable in the elution buffer for at least 30 days.

3. An elution buffer according to claim 1, wherein at least one mass spectrometry m/z peak value of the ionisation control protein is substantially stable for at least 30 days.

4. An elution buffer according to claim 1, wherein the ionisation control protein is selected to have at least one mass spectrometry peak having an m/z value which does not substantially overlap with a mass spectrometry peak of the or each predetermined analyte.

5. An elution buffer according to claim 4, wherein the ionisation control protein is selected to have at least one mass spectrometry m/z peak value within a predetermined mass spectrometry window used for the detection or quantification of one or more peaks from the at least one predetermined analyte.

6. An elution buffer according to claim 1 comprising an elution buffer selected from:
(a) 5% v/v acetic acid in water;
(b) 0.1 glycine, pH 2.0-3.0, or 0.2 M glycine pH 2-6.

7. An elution buffer according to claim 1 comprising 0.5 to 100 ng of ionisation control protein.

8. An elution buffer according to claim 1, wherein the ionisation control protein comprises at least 30 amino acids.

9. An elution buffer according to claim 1, wherein the ionisation control protein has a mass of at least 3 kDa.

10. An elution buffer according to claim 1, wherein the ionisation control protein is selected from aprotinin, β2 glycoprotein, transthyretin and α1 acid glycoprotein.

11. A kit for use in the analysis by mass spectrometry of one or more analytes comprising an elution buffer according to claim 1 and one or more analyte specific antibodies or fragments thereof specific for the one or more predetermined analytes.

12. A kit according to claim 11, wherein the analyte is a protein or peptide.

13. A kit according to claim 11, wherein the analyte or antigen specific antibody is a serum protein or peptide.

14. A kit according to claim 11, wherein the serum protein is a complement protein, an immunoglobulin or fragment thereof, albumin, β2 microglobulin, α1 microglobulin, cystatin C, a microalbumin, α1 acid glycoprotein, α1 antitrypsin, α2-macroglobin, anti-streptolysin-O, anti-tetanus toxoid immunoglobulin, apolipoprotein A, apolipoprotein B, caeruloplasmin, C-reactive protein, haptoglobin, prealbumin, rheumatoid factor, total serum protein transferrin, Haemophilia influenzae-specific immunoglobulin, diptheria toxoid-specific immunoglobulin, Streptococcus pneumoniae specific immunoglobulin, Salmonella typhi-specific immunoglobulin or Varicella zoster virus-specific immunoglobulin.

15. A kit according to claim 14, wherein the analyte specific antibodies are anti-IgA, anti-IgG, anti-IgD, anti-IgD, anti-IgE, anti-total light chain, anti-free light chain, anti-lambda light chain, anti-kappa light chain, anti-lambda free light chain, anti-kappa free light chain, anti-heavy chain subclass, anti-heavy chain class-light chain type or anti-heavy chain subclass-light chain type specific.

16. A kit according to claim 11, wherein the antibodies or fragments thereof are bound to a substrate.

17. A kit according to claim 11 comprising anti-IgG, anti-IgA, anti-IgM, anti-kappa and/or anti-lambda-specific antibodies.

18. A kit according to claim 14 comprising a predetermined amount of a control analyte.

19. A kit according to claim 11 comprising one or more of a sample diluent buffer, a reducing agent, a mass spectrometry matrix, a mass spectrometry matrix solvent, a MALDI target and a mass spectrometer mass calibrator.

20. A kit according to claim 11, additionally comprising a standard serum protein control.

21. A method of detecting or quantifying an analyte comprising immunopurifying a predetermined analyte, eluting the analyte with an elution buffer according to claim 1 and detecting the analyte and the ionisation control protein by mass spectrometry.

22. A method according to claim 21 wherein the mass spectrometry is MALDI-TOF.

23. A method according to claim 21 comprising the use of a kit.

24. A method of producing an elution buffer according to claim 1, comprising:
(a) identifying the analyte;
(b) identifying the m/z of at least one peak for the ionisation control compared to the m/z of one or more expected peaks for the analyte;
(c) identifying ionisation control proteins having the m/z range and acid stability.

25. A computer implemented method comprising imputing an analyte, comparing one or more m/z peaks for the analyte with the m/z peak of a plurality of potential ionisation control proteins having acid stability, and outputting the identification of one or more ionisation control proteins having the m/z range and acid stability for the analyte.

26. A method according to claim 25, wherein the computer comprises a computer processor and a computer memory.

27. An apparatus for analysis by mass spectrometry of one or more analytes a method according to claim 21, comprising the use of a computer implemented method comprising imputing an analyte, comparing one or more m/z peaks for the analyte with the m/z peak of a plurality of potential ionisation control proteins having acid stability, and outputting the identification of one or more ionisation control proteins having the m/z range and acid stability for the analyte.

28. An apparatus according to claim 27, comprising a mass spectrometer.
